(19) United States
(12) Patent Application Publication
(10) Pub. No.: US 2005/0250728 A1
(43) Pub. Date: Nov. 10, 2005

(54) Enhancing the efficacy of reverse transcriptase and dna polymerase inhibitors (nucleoside analogs) using pnp inhibitors and/or 2'-deoxyguanosine and/or prodrug thereof

(76) Inventors: Shanta Bantia, Birmingham, AL (US); Yarlagadda S. Babu, Birmingham, AL (US)

Correspondence Address: CONNOLLY BOVE LODGE & HUTZ LLP, SUITE 800, 1990 M STREET NW, WASHINGTON, DC, 20036-3425, US

(21) Appl. No.: 10/515,229

(22) PCT Filed: May 23, 2003
(86) PCT No.: PCT/US03/16364

§ 371 Date: Jun. 7, 2005

Related U.S. Application Data
(60) Provisional application No. 60/382,371, filed on May 23, 2002.

(51) Int. Cl. A61K 31/7076

(52) U.S. Cl. 514/45

(57) ABSTRACT

The efficacy of reverse transcriptase and DNA polymerase inhibitors (nucleoside analogs) in a mammalian host is enhanced by administering an effective amount of PNP inhibitor of prodrug of PNP inhibitor and/or an effective amounts of 2′-de-oxyguanosine and/or prodrugs thereof.

### FIELD OF THE INVENTION

The present invention relates to enhancing the efficacy of reverse transcriptase and DNA polymerase inhibitors (nucleoside analogs) in cancer and viral diseases using at least one of PNP inhibitors, prodrugs thereof, 2′-deoxyguanosine, prodrugs thereof and mixtures.

### BACKGROUND OF INVENTION

PNP deficient children exhibit a selective depletion of T-cells (1-3% of normal) and their T-cells response to mitogenic and allogenic stimuli are severely compromised suggesting that PNP activity may be required for normal human T-cell proliferation. PNP deficient children have an increase in deoxyguanosine (dGuo) levels, both in plasma and urine, as well as elevated concentrations of dGTP in red cells. Normally dGuo is converted by PNP to guanine. Deficiency or PNP inhibition results in accumulation of dGuo in plasma. DGuo is metabolized to dGMP by deoxycytidine (dCyd) kinase and dGMP is further converted to dGTP by other cellular kinases. The intracellular level of dGTP is important, as it has been demonstrated that higher concentrations of dGTP inhibit T-cell proliferation. It has been proposed that increased dGTP levels inhibit ribonucleotide reductase leading to inhibition of the synthesis of dCTP and dTTP. Depletion of dCTP and dTTP leads to inhibition of DNA synthesis and cell death. Nucleoside analogs such as 3TC, AZT, d4T, and ddc are routinely used for the treatment of cancer, HIV, and hepatitis B infection. The antiviral and anticancer effect of these drugs is due to incorporation of the triphosphate form of these nucleosides into the gene and causing chain termination. The efficacy of these drugs can be further enhanced if the competing nucleotide (dCTP/dTTP) pool is decreased further. Thus PNP inhibitors in the presence of dGuo should potentiate the activity of thymidine and deoxycytidine derived analogs such as 3TC, d4T, ddc, and AZT.

### SUMMARY OF INVENTION

According to the present invention, the efficacy of reverse transcriptase and DNA polymerase inhibitors (nucleoside analogs) is enhanced by decreasing the competing nucleotide (dCTP/dTTP) pool further, using PNP inhibitors and/or deoxyguanosine. In particular, the present invention relates to a process for enhancing the efficacy of these nucleoside analogs in a mammalian host being treated with a nucleoside analog that comprises administering to said host an effective amount of PNP inhibitor and/or prodrug of PNP inhibitor and/or an effective amount of 2′-deoxyguanosine and/or prodrug of 2′-deoxyguanosine. The PNP inhibitor typically has a Ki of 50 nanomolar or less.

Still other objects and advantages of the present invention will become readily apparent by those skilled in the art from the following detailed description, wherein it is shown and described only the preferred embodiments of the invention. As will be realized the invention is capable of other and different embodiments, and its several details are capable of modifications in various respects, without departing from the invention. Accordingly, the description is to be regarded as illustrative in nature and not as restrictive.

### BEST AND VARIOUS MODES FOR CARRYING OUT INVENTION

The present invention relates to enhancing the efficacy of reverse transcriptase and DNA Polymerase inhibitors (nucleoside analog) in a mammalian host being treated with a nucleoside analog. Typical nucleoside analogs 3TC, AZT, ddc, and d4T are routinely used for treating cancer, HIV, and/or hepatitis B infection.

The process of the present invention comprises administering to the mammalian host and especially a human host an effective amount of at least one PNP inhibitor or a prodrug of PNP inhibitor and/or an effective amount of 2′-deoxyguanosine and/or prodrug of 2′-deoxyguanosine. The PNP inhibitor employed according to the present invention typically has a Kᵢ value of about 50 nanomolar or less and preferably about 1 picomolar or less.

Examples of suitable PNP inhibitors employed according to the present invention are those disclosed in U.S. Pat. Nos. 4,985,433; 4,985,434; 5,008,265; 5,008,270; 5,565,463; 5,721,240 and 6,458,799 and U.S. patent applications Ser. Nos. 09/813,832 and 10/016,108 assigned to BioCryst Pharmaceuticals, Inc.; U.S. Pat. Nos. 5,985,848, 6,066,722 and 6,228,741; PCT Applications PCT/NZ00/00048 and PCT/NZ01/00174 disclosures of which are incorporated herein by reference.

Examples of some specific PNP inhibitors are:
1. (1S)-1,4-dideoxy-1-C-(4-hydroxypyrrolo[3,2-d]pyrimidin-7-yl)-1,4-imino-D-ribitol
2. (1S)-1-C-(2-amino-4-hydroxypyrrolo[3,2d]pyrimidin-7-yl)-1,4-dideoxy-1,4-imino-D-ribitol
3. (1R)-1-C-(4-hydroxypyrrolo[3,2-pyrimidin-7-yl)-1,4-imino-1,2,4-trideoxy-D-erythro-pentitol
4. (1S)-1-C-(4-hydroxypyrrolo[3,2-d]pyrimidin-7-yl)-1,4-imino-1,4,5-trideoxy-D-ribitol
5. (1S)-1,4-dideoxy-1-C-(4-hydroxypyrrolo[3,2-d]pyrimidin-7-yl)-1,4-imino-5-methylthio-D-ribitol
6. (1S)-1,4-dideoxy-1-C-(2,4-dihydroxypyrrolo[3,2-pyrimidin-7-yl)-1,4-imino-D-ribitol
7. (1R)-1-C-(2,4-dihydroxypyrrolo[3,2-d]pyrimidin-7-yl)-1,4-imino-1,2,4-trideoxy-D-erythro-pentitol
8. (1S)-1-C-(2,4-dihydroxypyrrolo[3,2-d]pyrimidin-7-yl)-1,4-imino-1,4,5-trideoxy-D-ribitol
9. (1S)-1,4-dideoxy-1-C-(2,4-dihydroxypyrrolo[3,2-d]pyrimidin-7-yl)-1,4-imino-5-methylthio-D-ribitol
10. (1R)-1-C-(2-amino-4-hydroxypyrrolo[3,2-d]pyrimidin-7-yl)-1,4-imino-1,2,4-trideoxy-D-erythro-pentitol
11. (1S)-1-C-(2-amino-4-hydroxypyrrolo[3,2-d]pyrimidin-7-yl)-1,4-imino-1,4,5-trideoxy-D-ribitol
12. (1S)-1-C-(2-amino-4-hydroxypyrrolo[3,2-d]pyrimidin-7-yl)-1,4-dideoxy-1,4-imino-5-methylthio-D-ribitol
13. (1S)-1,4-dideoxy-1-C-(7-hydroxypyrazolo[4,3-d]pyrimidin-3-yl)-1,4-imino-D-ribitol
14. (1R)-1-C-(7-hydroxypyrazolo[4,3-d]pyrimidin-3-yl)-1,4-imino-1,2,4-trideoxy-D-erythro-pentitol
15. (1S)-1-C-(7-hydroxypyrazolo[4,3-d]pyrimidin-3-yl)-1,4-imino-1,4,5-trideoxy-D-ribitol
16. (1S)-1,4-dideoxy-1-C-(7-hydroxypyrazolo[4,3-d]pyrimidin-3-yl)-1,4-imino-5-methylthio-D-ribitol
17. (1S)-1,4-dideoxy-1-C-(5,7-dihydroxypyrazolo[4,3-d]pyrimidin-3-yl)-1,4-imino-D-ribitol
18. (1R)-1-C-(5,7-dihydroxypyrazolo[4,3-d]pyrimidin-3-yl)-1,4-imino-1,2,4-trideoxy-D-erythro-pentitol
19. (1S)-1-C-(5,7-dihydroxypyrazolo[4,3-d]pyrimidin-3-yl)-1,4-imino-1,4,5-trideoxy-D-ribitol
20. (1S)-1,4-dideoxy-1-C-(5,7 dihydroxypyrazolo[4,3-d]pyrimidin-3-yl)-1,4-imino-5-methylthio-D-ribitol
21. (1S)-1-C-(5-amino-7-hydroxypyrazolo[4,3-d]pyrimidin-3-yl)-1,4-dideoxy-1,4-imino-D-ribitol
22. (1R)-1-C-(5-amino-7-hydroxypyrazolo[4,3-d]pyrimidin-3-yl)-1,4-imino-1,2,4-trideoxy-D-erythro-pentitol
23. (1S)-1-C-(5-amino-7-hydroxypyrazolo[4,3-d]pyrimidin-3-yl)-1,4-imino-1,4,5-trideoxy-D-ribitol
24. (1S)-1-C-(5-amino-7-hydroxypyrazolo[4,3-d]pyrimidin-3-yl)-1,4-dideoxy-1,4-imino-5-methylthio-D-ribitol
25. (1S)-1-C-(3-amino-2-carboxamido4-pyrrolyl )-1,4-dideoxy-1,4-imino-D-ribitol
26. (1S)-1,4-dideoxy-1-C-(4-hydroxypyrrolo[3,2-d]pyrimidin-7-yl)-1,4-imino-D-ribitol 5-phosphate
27. (1S)-1-C-(2-amino-4-hydroxypyrrolo[3,2-d]pyrimidin-7-yl)-1,4-imino-D-ribitol 5-phosphate
28. (1S)-1-C-(3-amino-2-carboxamido-4-15-pyrrolyl)-1,4-dideoxy-1,4-imino-D-ribitol
Preferred are compounds Ib and Ic, their tautomers and pharmaceutically acceptable salts.

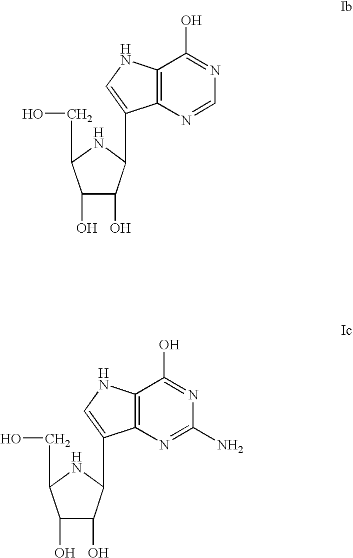

The most preferred PNP inhibitor employed according to the present invention is (1S)-1-(9-deazahypoxanthin-9-yl)-1,4dideoxy-1,4-imino-D-ribitol.

Examples of suitable prodrugs of 2′-deoxyguanosine are represented by the following: 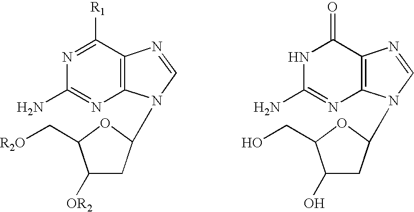
Wherein R₁ is Cl, NH₂, NHCH₃, R₃O, R₃S, or H;
   R₂ is acyl typically having 1 to 6 carbon atoms, and
   R₃ is alkyl typically having 1 to 3 carbon atoms and more typically 1 carbon atom.

The first five structural types (i.e., R₁ is Cl, NH₂, NHCH₃, R₃O, or R₃S) are converted to 2′-deoxyguanosine in vivo by esterases and adenosine deaminase.

The sixth type (R₁═H) is oxidized in vivo to 2′-deoxyguanosine. Examples of these in vivo conversions are discussed in Montgomery, Prog. In Med. Chem. 7, 69 (1970) and Jones, Antiviral Chemistry and Chemotherapy 9, 283 (1998). Mixtures of prodrugs can be employed, if desired, as well as mixtures of one or more prodrugs with 2′-deoxyguanosine.

When both the PNP inhibitor and/or prodrug thereof and the 2′-deoxyguanosine or prodrug thereof are used, the PNP inhibitor and/or prodrug thereof is typically administered prior to or at the same time as the 2′-deoxyguanosine and/or prodrug of 2′-deoxyguanosine. The PNP inhibitor is typically present in the host's bloodstream with 2′-deoxyguanosine in order to effectively prolong the half-life of the 2′-deoxyguanosine to permit a sufficient accumulation of 2′-deoxyguanosine triphosphate in T-cells to inhibit ribonucleotide reductase and thus decrease the other deoxynucleotides. When the PNP inhibitor is administered prior to the 2′-deoxyguanosine and/or prodrug of 2′deoxyguanosine, it is typically administered up to about 1 hour prior to the 2′-deoxyguanosine and/or prodrug of 2′-deoxyguanosine.

The compounds of the present invention can be administered by any conventional means available for use in conjunction with pharmaceuticals. They can be administered alone, but generally administered with a pharmaceutical carrier selected on the basis of the chosen route of administration and standard pharmaceutical practice.

The compounds of the present invention are typically administered to the patient within 24 hours prior to treatment with the nucleoside analog, more typically within about 12 hours prior to treatment with the nucleoside analog and preferably simultaneously or substantially simultaneously along with the nucleoside analog.

The dosage administered will, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to 1000 milligram (mg) per kilogram (kg) of body weight, with the preferred dose being 0.1 to about 30 mg/kg.

The amount of PNP inhibitors and/or prodrug thereof and/or 2′-deoxyguanosine and/or prodrug thereof is typically sufficient to increase the intracellular amounts of dGTP by 5-fold and more typically by about 10-fold.

Dosage forms (compositions suitable for administration) contain from about 1 mg to about 100 mg of active ingredient per unit. In these pharmaceutical compositions, the active ingredient will ordinarily be present in an amount of about 0.5-95% by weight based on the total weight of the composition.

The active ingredient can be administered orally in solid dosage forms, such as capsules, tablets, and powders, or in liquid dosage forms, such as elixirs, syrups, and suspensions. It can also be administered intravenously, subcutaneously, parenterally, in sterile liquid dosage forms. The active ingredient can also be administered intranasally (nose drops) or by inhalation. Other dosage forms are potentially possible such as administration transdermally, via a patch mechanism or ointment.

Gelatin capsules contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar-coated or film-coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and, if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propylparaben, and chlorobutanol.

Suitable pharmaceutical carriers are described in Remington's Pharmaceutical Sciences, Mack Publishing Company, a standard reference text in this field.

Useful pharmaceutical dosage forms for administration of the compounds according to the present invention can be illustrated as follows:

### Capsules

A large number of unit capsules are prepared by filling standard two-piece hard gelatin capsules each with 100 mg of powdered active ingredient, 150 mg of lactose, 50 mg of cellulose, and 6 mg of magnesium stearate.

### Soft Gelatin Capsules

A mixture of active ingredient in a digestible oil such as soybean oil, cottonseed oil, or olive oil is prepared and injected by means of a positive displacement pump into gelatin to form soft gelatin capsules containing 100 mμ of the active ingredient. The capsules are washed and dried.

### Tablets

A large number of tablets are prepared by conventional procedures so that the dosage unit was 100 mg of active ingredient, 0.2 mg of colloidal silicon dioxide, 5 mg of magnesium stearate, 275 mg of microcrystalline cellulose, 11 mg of starch, and 98.8 mg of lactose. Appropriate coatings may be applied to increase palatability or delay absorption.

Moreover, the compounds of the present invention can be administered in the form of nose drops or a nasal inhaler.

Various modifications of the invention in addition to those shown and described herein will be apparent to those skilled in the art from the foregoing description. Such modifications are also intended to fall within the scope of the appended claims.

The foregoing disclosure includes all the information deemed essential to enable those skilled in the art to practice the claimed invention. Because the cited applications may provide further useful information, these cited materials are hereby incorporated by reference in their entirety.

The foregoing description of the invention illustrates and describes the present invention. Additionally, the disclosure shows and describes only the preferred embodiments of the invention but, as mentioned above, it is to be understood that the invention is capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. Also, it is intended that the appended claims be construed to include alternative embodiments.

1. A process for enhancing the efficacy of reverse transcriptase and DNA polymerase inhibitors (nucleoside analog) in a mammalian host being treated with a nucleoside analog which comprises administering to said host an effective amount of at least one member selected from the group consisting of at least one PNP inhibitor, prodrug of PNP inhibitor, 2′-deoxyguanosine, prodrug of 2′-deoxyguanosine and mixture thereof.

2. The process of claim 1 wherein the PNP inhibitor is administered simultaneously with the at least one member or prior to the at least one member.

3. The process of claim 2 wherein the PNP inhibitor is administered up to about 1 hour prior to administering the at least one member.

4. The process of claim 1 which comprises orally administering the inhibitor.

5. The process of claim 4 which comprises administering the at least one member by infusion.

6. The process of claim 4 which comprises orally administering the at least one member.

7. The process of claim 1 wherein the at least one member is 2′-deoxyguanosine.

8. The process of claim 3 wherein the at least one member is 2′-deoxyguanosine.

9. The process of claim 6 wherein the at least one member is 2′-deoxyguanosine.

10. The process of claim 3 wherein the PNP inhibitor is administered up to about 1 hour prior to administering the at least one member.

11. The process of claim 3 which comprises orally administering the inhibitor.

12. The process of claim 1 which comprises administering said at least one member and said inhibitor intravenously.

13. The process of claim 1 which comprises administering said at least one member and said inhibitor subcutaneously.

14. The process of claim 1 wherein the PNP inhibitor comprises (1S)-1-(9-deazahypoxanthin-9-yl)-1,4dideoxy-1,4imino-D-ribitol.
